SEQUENCE LISTING (30 sequences)

SEQ ID NO: 1 - DNA, 55 bp, Artificial Sequence
  Description of Artificial Sequence Synthetic oligonucleotide
cgaagagatccctgaggaatacaaccgtctgtgcatcaaaccgcgtgactggatc

SEQ ID NO: 2 - DNA, 62 bp, Artificial Sequence
  Description of Artificial Sequence Synthetic oligonucleotide
gcattcgtcgatccagtcacgcggtttgatgcacagacggttgtattcctcagggatctctt

SEQ ID NO: 3 - DNA, 80 bp, Artificial Sequence
  Description of Artificial Sequence Synthetic oligonucleotide
gaatgcgactccaacgaaggtggtgaacgtgcttacttccgtaacggtaaaggtggttgcgattccttctggatctgccc

SEQ ID NO: 4 - DNA, 80 bp, Artificial Sequence
  Description of Artificial Sequence Synthetic oligonucleotide
tcttccgggcagatccagaaggaatcgcaaccacctttaccgttacggaagtaagcacgttcaccaccttcgttggagtc

SEQ ID NO: 5 - DNA, 67 bp, Artificial Sequence
  Description of Artificial Sequence Synthetic oligonucleotide
ggaagaccacaccggtgctgactactactcctcctaccgtgactgcttcaacgcttgcatctaatga

SEQ ID NO: 6 - DNA, 65 bp, Artificial Sequence
  Description of Artificial Sequence Synthetic oligonucleotide
agcttcattagatgcaagcgttgaagcagtcacggtaggaggagtagtagtcagcaccggtgtgg

SEQ ID NO: 7 - DNA, 205 bp, Artificial Sequence
  Description of Artificial Sequence Synthetic oligonucleotide
cgaagagatccctgaggaatacaaccgtctgtgcatcaaaccgcgtgactggatcgacgaatgcgactccaacgaaggtggtgaacgtgcttacttccgtaacggtaaaggtggttgcgattccttctggatctgcccggaagaccacaccggtgctgactactactcctcctaccgtgactgcttcaacgcttgcatctaatga

SEQ ID NO: 8 - DNA, 70 bp, Artificial Sequence
  Description of Artificial Sequence Synthetic oligonucleotide
cgaagagatccctgaggaataccttcaggatccctacaaccgtctgtgcatcaaaccgcgtgactggatc

SEQ ID NO: 9 - DNA, 77 bp, Artificial Sequence
  Description of Artificial Sequence Synthetic oligonucleotide
gcattcgtcgatccagtcacgcggtttgatgcacagacggttgtagggatcctgaaggtattcctcagggatctctt

SEQ ID NO: 10 - DNA, 73 bp, Artificial Sequence
  Description of Artificial Sequence Synthetic oligonucleotide
cgaagagatccctgaggaatacgctatcgaaggtcgttacaaccgtctgtgcatcaaaccgcgtgactggatc

SEQ ID NO: 11 - DNA, 80 bp, Artificial Sequence
  Description of Artificial Sequence Synthetic oligonucleotide
gcattcgtcgatccagtcacgcggtttgatgcacagacggttgtaacgaccttcgatagcgtattcctcagggatctctt

SEQ ID NO: 12 - DNA, 82 bp, Artificial Sequence
  Description of Artificial Sequence Synthetic oligonucleotide
gacgaatgcgactccaacgaaggtggtgaacgtgcttacttccgtaacggtaaaggtggttgcggttccttctggatctgcc

SEQ ID NO: 13 - DNA, 80 bp, Artificial Sequence
  Description of Artificial Sequence Synthetic oligonucleotide
tcttccgggcagatccagaaggaaccgcaaccacctttaccgttacggaagtaagcacgttcaccaccttcgttggagtc

SEQ ID NO: 14 - DNA, 195 bp, Artificial Sequence
  Description of Artificial Sequence synthetic DNA sequence coding for Refludan (Leu - hirudin)
cttacgtatactgactgcactgaatctggtcagaacctgtgcctgtgcgaaggatctaacgtttgcggccagggtaacaaatgcatccttggatccgacggtgaaaagaaccagtgcgttactggcgaaggtaccccgaaaccgcagtctcataacgacggcgacttcgaagagatccctgaggaataccttcag

SEQ ID NO: 15 - protein, 65 aa, Artificial Sequence
  Description of Artificial Sequence Synthetic Amino acid sequence of Refludan (Leu - hirudin)
LTYTDCTESGQNLCLCEGSNVCGQGNKCILGSDGEKNQCVTGEGTPKPQSHNDGDFEEIPEEYLQ

SEQ ID NO: 16 - DNA, 180 bp, Artificial Sequence
  Description of Artificial Sequence synthetic DNA sequence coding for TAP
tacaaccgtctgtgcatcaaaccgcgtgactggatcgacgaatgcgactccaacgaaggtggtgaacgtgcttacttccgtaacggtaaaggtggttgcgattccttctggatctgcccggaagaccacaccggtgctgactactactcctcctaccgtgactgcttcaacgcttgcatc

SEQ ID NO: 17 - protein, 60 aa, Artificial Sequence
  Description of Artificial Sequence synthetic amino acid sequence for TAP
YNRLCIKPRDWIDECDSNEGGERAYFRNGKGGCDSFWICPEDHTGADYYSSYRDCFNACI

SEQ ID NO: 18 - protein, 30 aa, Unknown Organism
  Description of Unknown Organism Control cgtase-Ala-hirudin
MKRNRFFNTSAAIAISIALNTFFCSMQTIA

SEQ ID NO: 19 - protein, 21 aa, Serratia marcescens
MKKTAIALAVALAGFATVAQA

SEQ ID NO: 20 - protein, 22 aa, Pseudomonas fluorescens
MKNTLGLAIGSLIAATSFGVLA

SEQ ID NO: 21 - protein, 25 aa, Escherichia coli
MMITLRKLPLAVAVAAGVMSAQAMA

SEQ ID NO: 22 - protein, 25 aa, Shewanella putrefaciens
MKKMNLAVCIATLMGTAGLMGTAVA

SEQ ID NO: 23 - protein, 23 aa, Unknown Organism
  Description of Unknown Organism B-Lactamase/pBR322
MSIQHFRVALIPFFAAFSLPVFA

SEQ ID NO: 24 - protein, 21 aa, Escherichia coli
MKQSTIALALLPLLFTPVTKA

SEQ ID NO: 25 - protein, 21 aa, Escherichia fergusonii
MKQSAIALALLSCLITPVSQA

SEQ ID NO: 26 - protein, 27 aa, Paenibacillus macerans
MKSRYKRLTSLALSLSMALGISLPAWA

SEQ ID NO: 27 - protein, 24 aa, Salmonella typhimurium
MSFHHRVFKLSALSLALFSHLSFA

SEQ ID NO: 28 - protein, 9 aa, Artificial Sequence
  Description of Artificial Sequence Synthetic peptide
EEYLQDPYN

SEQ ID NO: 29 - protein, 6 aa, Artificial Sequence
  Description of Artificial Sequence Synthetic peptide
AIEGRY

SEQ ID NO: 30 - DNA, 207 bp, Artificial Sequence
  Description of Artificial Sequence Synthetic oligonucleotide
agcttcattagatgcaagcgttgaagcagtcacggtaggaggagtagtagtcagcaccggtgtggtcttccgggcagatccagaaggaatcgcaaccacctttaccgttacggaagtaagcacgttcaccaccttcgttggagtcgcattcgtcgatccagtcacgcggtttgatgcatagacggttgtattcctcagggatctctt

(19) United States
(12) Patent Application Publication
(10) Pub. No.: US 2002/0173620 A1
(43) Pub. Date: Nov. 21, 2002

(54) Bifunctional fusion proteins formed from hirudin and TAP

(76) Inventor: Paul Habermann, Eppstein (DE)

Correspondence Address: FINNEGAN, HENDERSON, FARABOW,, GARRETT AND DUNNER, Franklin Square Bldg., Suite 700, 1300 I Street, N.W., Washington, DC, 20005-3315, US

(21) Appl. No.: 09/899,235

(22) Filed: Jul. 6, 2001

(30) Foreign Application Priority Data
Jul. 7, 2000 (DE) 10033195.5-41

(51) Int. Cl. C12N 9/64

(52) U.S. Cl. 530/350; 435/226

(57) ABSTRACT

The invention relates to bifunctional fusion proteins, which contain hirudin or a variant of hirudin and TAP (tick anticoagulant peptide) or a variant of TAP; the preparation and use of these bifunctional fusion proteins; and pharmaceutical compositions comprising these bifunctional fusion proteins.

The invention relates to bifunctional fusion proteins, which contain hirudin or a variant of hirudin and TAP (tick anticoagulant peptide) or a variant of TAP; the preparation and use of these bifunctional fusion proteins; and pharmaceutical compositions comprising these bifunctional fusion proteins.

The complexity of the human blood clotting system requires the involvement of a large number of blood clotting factors. These clotting factors can originate from protein families whose members have common structural properties, but that on account of slight structural changes developed during evolution, act by interaction with another target structure that is specific to the particular clotting factor in question. On account of the structural relationship of the members of these protein families and the specificity developed during evolution, it is very difficult to find chemically-synthesized molecules which, on one hand interact highly specifically with one of the members, but on the other hand do not interact with any of the other members of the family so that upon therapeutic administration the risk of side effects is minimized.

Over hundreds of millions of years blood sucking parasites have developed peptides that interact specifically with blood clotting factors and thereby only slightly damage the host. In the course of this evolution, host/parasite-specific mechanisms of inhibition of clotting were developed.

Leaches of the type Hirudo developed, for example, various isoforms of the thrombin inhibitor hirudin. By means of synthetic variation of the molecule, e.g. replacement of the N-terminal amino acid, hirudin has been optimized for pharmaceutical technology requirements (e.g. European Patent No. EP 0 324 712.)

Other leaches such as Hementeria gigantii developed proteins that dissolve blood clots and act similarly to human tPA (tissue plasminogen activator). Tuszynski et al., J. of Biol. Chem. 262:9718-9723 (1987), describe a protein about 17 000 dt in size, which can be isolated from the Mexican leach and is an inhibitor of blood clotting factor Xa.

Ticks have also developed thrombin inhibitors. European Patent No. EP 0 345 616 describes the protein amblyommin, which can be isolated from African hard ticks. Amblyommin inhibits thrombin, although it has a primary structure different from hirudin.

This shows that with the same target protein, nevertheless, various inhibitory proteins that have a similar action have been developed during evolution. Soft ticks such as Ornithodoros moubata have developed inhibitors of blood clotting factor Xa. The polypeptide TAP (tick anticoagulant peptide), Waxman L. et al., Science 248:595-596 (1990) specifically inhibits the blood clotting factor Xa, which converts inactive prothrombin into active thrombin. U.S. Pat. Nos. 5,239,058 and 5,328,997 describe TAP and its preparation.

These examples show that various stages in the blood-clotting cascade (various blood clotting factors) have been developed as a target of the inhibition of clotting during evolution and that the mechanism of inhibition has been specifically optimized by individual animal species according to the individual living circumstances.

Proteins that combine the properties of various of these inhibitors so that synthetic bifunctional proteins result are of great pharmaceutical interest.

Seno et. al., FEBS Letters 199:187-192 (1986), and European Patent No. EP 0 288 809 describe the principle of synthetic bifunctional proteins. The basis of these proteins are polypeptides that are immunologically active and originate from the group consisting of lymphokines and interferons. For the construction of fusion proteins, European Patent No. EP 0 227 938 describes the use of a factor Xa cleavage site as a connecting link between interleukin-2 and a second subprotein, which is preferably proinsulin or hirudin. The protein of interest can be separated by means of the factor Xa cleavage site. Interleukin-2 shows no interleukin-2 activity in the fusion protein.

European Patent No. EP 0 502 968 describes albuminous plasminogen analogues, which can be cleaved by an enzyme involved in blood clotting, such as factor Xa, whereby a compound having plasmin activity is formed. Firstly, inactive fusion proteins, consisting, for example, of hirudin and streptokinase, are fused by means of this cleavage site and, after injection into the blood circulation system, are cleaved by enzymes of the clotting cascade, e.g. by the factor Xa, by means of which the form of the subprotein is formed. The subprotein retains activity, for example, in the case of hirudin and streptokinase, inhibition of blood clot formation by hirudin and fibrinolysis by streptokinase occurs. The fusion proteins act as a ‘prodrug’; i.e. they must first be converted into their active form by cleavage. As a prodrug, they are inactive or only active to a slight extent. This situation has the disadvantage, inter alia, that these fusion proteins do not act immediately, but only after their activation or cleavage. Additionally, these fusion proteins are not active to such an extent that the danger of blood clotting is suppressed. This situation is undesirable, for example, in the case of a massive injury such as the formation of thrombi after an operation.

It is an object of the present invention to prepare a bifunctional fusion protein, both subproteins being active even in the fusion protein and both subproteins inhibiting blood clotting.

The invention relates to a bifunctional fusion protein, which contains hirudin, a variant of hirudin, and TAP or a variant of TAP.

Surprisingly, it has been found that using hirudin and the factor Xa inhibitor peptide isolated from soft ticks, tick anticoagulant peptide (hereinafter “TAP”), fusion proteins can be formed which are bifunctionally active, i.e. both subproteins of the bifunctional fusion protein—hirudin and TAP—are active even without prior enzymatic activation or cleavage.

Since both functions—on one hand hirudin or a variant of hirudin, and on the other hand TAP or a variant of TAP—are active even in the fusion protein, a cleavage to the subproteins—i.e. to hirudin or a variant of hirudin on one hand and to TAP or a variant of TAP on the other hand—is not a necessary requirement for the activity of the subproteins.

In one embodiment of the invention, in these synthetic bifunctional fusion proteins, each individual property or function of the subproteins is comparable in efficiency to those of the respective starting proteins. Therefore, for pharmaceutical administration, the subproteins do not need to be separated or administered as a mixture. The bifunctional proteins have improved half-lives, so that less of each subprotein needs to be administered compared to the case of administration of the individual proteins. An advantage is that the risk of hemorrhage can be reduced thereby.

Moreover, the two subproteins interact with various targets of the blood-clotting cascade. This has the advantage that in acute situations, clot formation is inhibited by hirudin, which inhibits thrombin, and simultaneously the regeneration of thrombin from prothrombin, which proceeds via the enzymatic activity of factor Xa, is prevented by TAP. The antithrombotic action is thereby extended and amplified.

The invention comprises the use of hirudin or hirudin variants in the bifunctional fusion protein. In one embodiment of the invention, one of the natural isoforms of hirudin (the natural isoforms are together hereinafter described as “hirudin”) is used. A natural isoform is, for example, Val-Val-hirudin. In other embodiments of the invention, a variant of a natural hirudin isoform is employed. A variant is derived from a natural isoform of hirudin, but contains, for example, additional amino acids and/or amino acid deletions and/or amino acid replacements in comparison with the natural isoform. A variant of hirudin can contain alternating peptide sections of natural isoforms of hirudin and new amino acids. For example, a variant of hirudin can link a natural sequence of up to 15 amino acids to a short, for example 6-20 amino acids long, new sequence. A hirudin variant can, in one embodiment of the invention, thereby be produced which contains no disulfide bridges. Variants of hirudin are known and described, for example, in German Patent No. DE 3 430 556. Another embodiment of the invention relates to the hirudin variant refludan, or Leu-hirudin, also described as [Leu¹, Thr²]-63-desulfatohirudin or lepirudin, which is described in sequence number 4 of European Patent No. EP-B 0 324 712 and in SEQ ID NO: 15. A further embodiment relates to hirudin variants having delayed action (e.g. PEG-hirudin, described in European Patent No. EP 0 345 616). Another embodiment of the invention relates to hirudin and hirudin variants, which, compared to the natural isoforms or variants, are truncated at the N terminus and/or C terminus. In another embodiment of the invention, a hirudin variant has 80% or more homology (amino acid identity) to a natural isoform of hirudin.

In an analogous manner, isoforms and variants of TAP can be used. In another embodiment of the invention, a natural isoform of TAP is used (natural isoforms are also included under the term “TAP”), e.g. TAP as in SEQ ID NO: 17 of the instant specification or as described by Waxman et al., Science 248:595-596 (1990). In other embodiments of the invention a variant of TAP is used. A variant of TAP is derived from a natural isoform of TAP, for example SEQ ID NO: 17, that contains, for example, additional amino acids and/or amino acid deletions and/or amino acid replacements (mutations) in comparison to the natural isoform. Another embodiment relates to TAP variants, which compared to the natural TAP isoform, TAP variants, or SEQ ID NO: 17, are truncated at the N terminus and/or C terminus. In other embodiments of the invention, a TAP variant has 80%, 90%, or more homology (amino acid identity) to a natural isoform of TAP, for example, SEQ ID NO: 17.

Another embodiment of the invention relates to a bifunctional fusion protein, in which the subproteins are connected to one another via a spacer. This spacer, in one embodiment of the invention, consists of one or more amino acids, for example up to 10 amino acids. Examples of spacers are: -Asp-Pro- and -Ala-Ile-Glu-Gly-Arg-.

Another embodiment of the invention relates to a bifunctional protein in which the two subproteins are linked via a factor Xa cleavage site. This embodiment has the advantage that an additional cleavage site, as a substrate in competition with the naturally occurring cleavage site in prothrombin, is offered to traces of factor Xa, which is not inactivated. By means of this substrate inhibition, a further advantageous concomitant effect is achieved. Surprisingly, neither the hirudin activity nor the TAP activity are adversely affected in this embodiment.

Examples of embodiments of the invention include the following fusion proteins:

Hir1-63TAP2-60

amino acids 1-63 of hirudin as in SEQ ID NO: 15, amino acids 2-60 of TAP as in SEQ ID NO: 17. Parts of the sequence are shown in FIG. 1 (SEQ ID NO: 7);

Hir₁₋₆₅-AspPro-TAP₁₋₆₀

fusion proteins comprising amino acids 1-65 of hirudin as in SEQ ID NO: 15, a spacer (Asp, Pro) and the amino acids 1-60 from TAP as in SEQ ID NO: 17;

Hir₁₋₆₃-Ala Ile Glu Gly Arg-TAP₁₋₆₀ (Gly 34)

amino acids 1-63 from hirudin as in SEQ ID NO: 15, a spacer (-Ala Ile Glu Gly Arg-) having a recognition site for factor Xa protease, and

amino acids 1-60 from TAP as in SEQ ID NO: 17, a glycine (Gly 34) being introduced into position 34 of the TAP sequence; and

Ala-Hir2-63-Ala Ile Glu Gly Arg-TAP1-60 (Gly 34)

amino acid Ala N terminus; amino acids 2-63 of hirudin as in SEQ ID NO: 15, spacer -Ala Ile Glu Gly Arg-, and amino acids 1-60 from TAP as in SEQ ID NO: 17. The fusion protein contains a glycine in position 34 of the TAP peptide sequence.

The invention also relates to a bifunctional fusion protein which contains one of the signal sequences SEQ ID NO: 18-27, for example SEQ ID NO: 18, SEQ ID NO: 19, SEQ ID NO: 20, SEQ ID NO: 21, or SEQ ID NO: 22 (Table 1, and German Patent Application No. DE 19 944 870.1).

The invention also relates to a nucleic acid, e.g. DNA, which codes for the bifunctional fusion protein. For example, the DNA contains one of the sequences SEQ ID NO: 14 (which codes for Leu-hirudin) and/or SEQ ID NO: 16 (which codes for TAP) or parts of these sequences. In other embodiments of the invention, the DNA additionally contains a sequence that codes for one of the signal sequences from Table 1.

In another embodiment of the invention, the fusion protein is encoded by a DNA that codes for hirudin or a hirudin variant and TAP or a TAP variant. The DNA coding for the bifunctional fusion protein is, in one embodiment of the invention present in a plasmid, e.g. the plasmid pK152 (European Patent No. EP 0 448 093; and European Patent Application No.8974322). The plasmid pK152 contains the sequence for hirudin as in European Patent No. EP 0 324 712. The invention further relates to a DNA that codes for the bifunctional fusion protein and a plasmid that contains this DNA. The invention also relates to the use of a DNA that codes for the bifunctional fusion protein and the use of a plasmid, which contains this DNA. In another embodiment of the invention, the plasmid also contains a promoter suitable for expression.

The invention also relates to a cell, which is eukaryotic or prokaryotic, and which contains a DNA that codes for the bifunctional fusion protein, e.g. said DNA being present in a plasmid. Another embodiment of the invention relates to a bacterial cell, e.g. E. coli. Yet another embodiment relates to a yeast cell, e.g. Saccharomyces cerevisiae.

The invention also relates to a process for the preparation of the bifunctional fusion protein. In one embodiment of the invention, the bifunctional fusion protein is prepared by heterologous expression in recombinant eukaryotic cells—e.g. yeast—or recombinant prokaryotic cells—e.g. E. coli, such as E. coli K12 MC 1061 (Sambrook et al. Molecular Cloning, Cold Spring Harbor Laboratory Press (1989).) It has been surprisingly found that a bifunctional fusion protein formed from hirudin and TAP can be expressed in E. coli at high yields, comparable to the expression of lepirudin, being released into the nutrient medium in gram amounts. This is surprising, since the bifunctional fusion protein contains up to 12 cysteine radicals that are important for biological function. TAP contains six cysteines, which can form three additional cysteine bridges. Both functional groups of the bifunctional fusion protein obtained by expression in E. coli are biologically active. The functional activity can be determined as in Example 1 (determination of the hirudin activity according to Grieβbach et al. Thrombosis Research 37:347-350 (1985)) and Example 2 (determination of the activity of TAP according to the method described in European Patent No. EP 0454372). It is also advantageous that the bifunctional fusion protein is expelled into the medium when expressed in E. coli, for example if one of the signal sequences from German Patent No. DE 19 944 870.1 is used.

Another embodiment of the invention relates to a process for the preparation of a bifunctional fusion protein in which the signal sequences described in German Patent Application No. DE 19 944 870.1 are used. German Patent Application No. DE 19 944 870.1 describes vectors and signal sequences that allow the expression and secretion of hirudin, hirudin variants, and bifunctional fusion proteins into the fermentation medium. European Patent No. EP 0 448 093 describes this process for the hirudin variant Ala-hirudin (Example 6). The biologically active bifunctional fusion protein can be isolated from this medium or cell supernatant. In one embodiment of the invention, the bifunctional fusion protein is prepared by fermenting the cells.

Since the bifunctional fusion protein is found in the medium or cell supernatant, the bifunctional fusion proteins can be prepared by means of inexpensive and simple processes, e.g. according to the process described in European Patent No. EP 0 549 915.

In other embodiments of the invention, the purified bifunctional fusion protein is freeze-dried.

The invention also relates to a bifunctional fusion protein comprising hirudin or a hirudin variant and TAP or a TAP variant, which fusion protein also contains a signal sequence, for example, one described in German Patent Application No. DE 19 944 870.1 (Table 1). Preferably, the fusion protein contains one of the signal sequences SEQ ID NO: 18, SEQ ID NO: 19, SEQ ID NO: 20, SEQ ID NO: 21, or SEQ ID NO: 22. The invention also relates to the use of the signal sequences from German Patent Application No. DE 19 944 870.1 or Table 1 for the preparation of the bifunctional fusion protein, the signal sequence being removed in the course of expression by E. coli.

The invention also relates to bifunctional fusion proteins that are coupled to a carrier. The coupling is, for instance, carried out via hirudin or the hirudin variant, for example as described in European Patent No. EP 0 345 616. The carrier used is, for example, PEG (polyethylene glycol) or dextran.

The invention further relates to the use of the bifunctional fusion proteins as pharmaceutical active compounds. The invention also relates to processes for the production of a pharmaceutical composition that contains the bifunctional fusion protein, for example, a process where the bifunctional fusion protein is first prepared, then mixed with a suitable pharmaceutical carrier, and, if appropriate, mixed also with further additives. The invention relates to a pharmaceutical composition that contains a bifunctional fusion protein, optionally further additives, and suitable pharmaceutical carriers. In one embodiment, the invention relates to a pharmaceutical composition prepared by mixing its components and subsequent freeze-drying. In another embodiment, the invention relates to a pharmaceutical composition for oral or nasal administration, for example, a nasal spray that contains a bifunctional fusion protein, and that can be used for, for example, the treatment of acute angina pectoris. The invention also relates to pharmaceutical composition that contain a bifunctional fusion protein bound to a carrier.

The bifunctional fusion protein or a pharmaceutical composition containing this bifunctional fusion protein can be used for the treatment and prevention of thrombotic events. These pharmaceutical compositions are also suitable, for example, for the prophylaxis of venous and arterial thromboses, for the prevention of consumption coagulopathy, or for the treatment of unstable angina pectoris.

### BRIEF DESCRIPTION OF THE FIGURES

FIG. 1: FIG. 1 shows the sequence of the DNA fragment that codes for the amino acids 57-63 from lepirudin and 2-60 from TAP.

FIG. 2: FIG. 2 shows the sequence of the oligonucleotides hir 65_DP_zehna 1 and hir 65_DP_zehna 2 needed, in addition to the oligonucleotides zehna 3 to zehna 6 from FIG. 1, for the construction of lepirudin₍₁₋₆₅₎-Asp, Pro- TAP₍1-60). The underlined sequence areas here mark the codons for the newly introduced amino acids.

FIG. 3: FIG. 3 shows the oligonucleotides needed, in addition to the sequences zehna 5/6 described in FIG. 1, for the construction of lepirudin₍₁₋₆₃₎-Ala, Ile, Glu, Gly, Arg-TAP₍₁₋₆₀₎.

FIG. 4: Representation of the DNA sequence (SEQ ID NO: 14) and amino acid sequence (SEQ ID NO: 15) of lepirudin (Leu-hirudin.) The sequence corresponds to the sequence from pk 152.

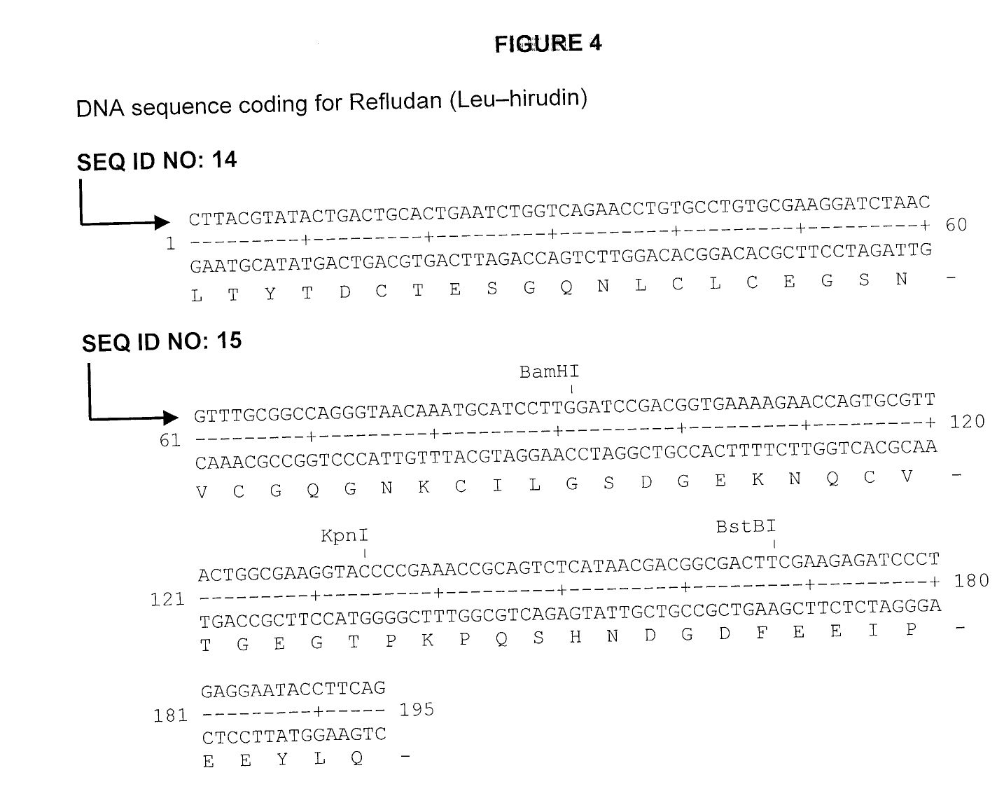

FIG. 5: Representation of the DNA sequence (SEQ ID NO: 16) and amino acid sequence (SEQ ID NO: 17) of TAP.

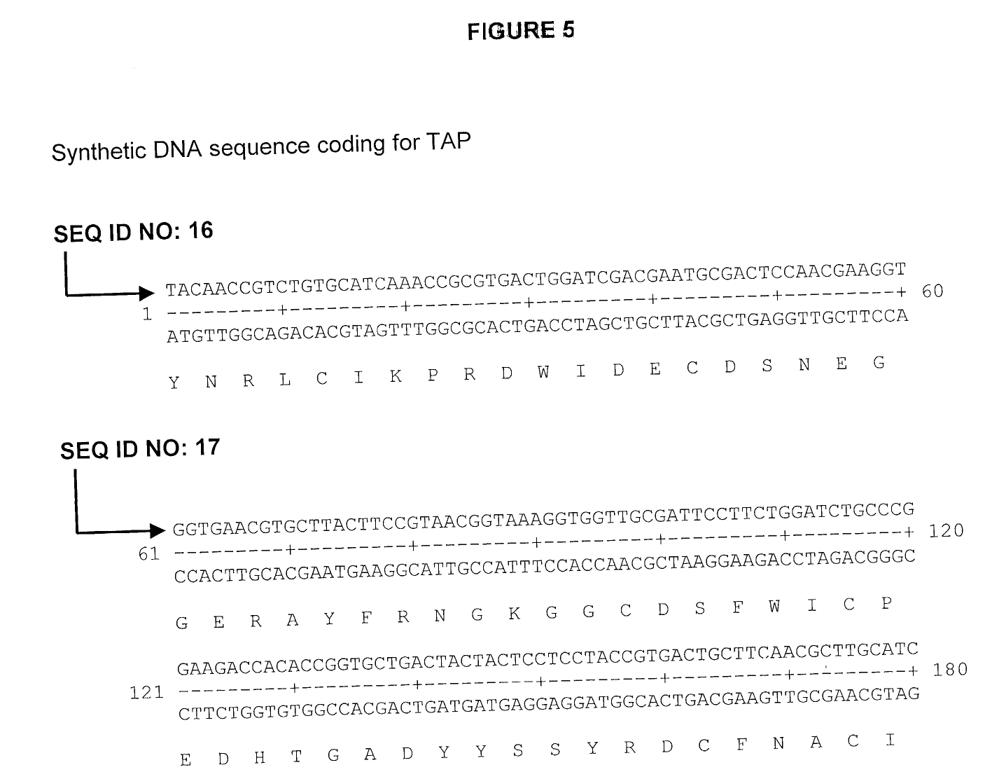

The underlined sequence sections mark the modifications described in each case.

### EXAMPLES

### Example 1

### Determination of the Hirudin Activity

The determination of the hirudin concentration was carried out according to the “Chromogenic assay” of Grieβbach et al., Thrombosis Research 37:347-350 (1985). For this purpose, defined amounts of a lepirudin standard solution (e.g. hirudin variant Leu-hirudin from European Patent No. EP 0 324 712) were assayed in order to plot a calibration curve. In this manner, the yield may be indicated directly in mg/l.

### Example 2

### Determination of the Activity of the Tick Anticoagulatory Peptide (TAP)

The verification of the expression of active TAP protein was carried out according to the description in European Patent No. EP 0 454 372 B1. The bacterial supernatants were diluted 1:5 for this purpose and then further diluted to give a dilution series with the following buffer: 50 mM Tris (pH 7.5-8.0), 150 mM NaCl, 0.1% BSA and 10% DMSO. The protein concentration was determined using hirudin or lepirudin. The molecular weight of the respective bifunctional fusion protein was used as a basis for the determination of the inhibition constant. This constant may be defined according to the description of Waxman et al., Science 248:595-596 (1990).

### Example 3

### Construction of Hir₁₋₆₃TAP₂₋₆₀ Hybrid (DNA that Codes for Fusion Protein Hirudin-TAP)

The plasmid pk152, which contains the sequence for hirudin as disclosed in European Patent No. EP 0 324 712, was used for the construction of the DNA sequence encoding this protein.

For the construction of the expression plasmid, the vector (laboratory name pD2B) described in German Patent Application No. DE 12 944 870.1 was used by way of example. The DNA sequence encoding the TAP was prepared synthetically. To this end, 6 oligonucleotide sequences SEQ ID NO: 1 to SEQ ID NO: 6 were prepared—e.g. with the aid of the Expedite Synthesis apparatus (PerSeptive Biosystems). These oligonucleotides have the following names and sequences:

SEQ ID NO: 1 (hir63_zehna1):
GluTyrAsn
5′- CGAAGAGATCCCTGAGGAATACAACCGTCTGTGCATCAAACCGCGTGACTGGATC -3′
SEQ ID NO: 2 (hir63_zehna2):
5′- GCATTCGTCGATCCAGTCACGCGGTTTGATGCACAGACGGTTGTATTCCTCAGGGATCTCTT -3′
SEQ ID NO: 3 (zehna3):
5′- GAATGCGACTCCAACCAAGGTCGTCAACGTGCTTACTTCCGTAACGGTAAAGGTGGTTGCGATTCCTTCTGGATCTGCCC -3′
SEQ ID NO: 4 (zehna4):
5′- TCTTCCGGGCAGATCCAGAAGGAATCGCAACCACCTTTACCGTTACGGAAGTAAGCACGTTCACCACCTTCGTTGGAGTC -3′
SEQ ID NO: 5 (zehna5):
5′- GGAAGACCACACCGGTGCTGACTACTACTCCTCCTACCGTGACTGCTTCAACGCTTGCATCTAATGA- 3′
SEQ ID NO: 6 (zehna6):
5′- AGCTTCATTAGATGCAAGCGTTGAAGCAGTCACGGTAGGAGGAGTAGTAGTCAGCACCGGTGTGG -3′

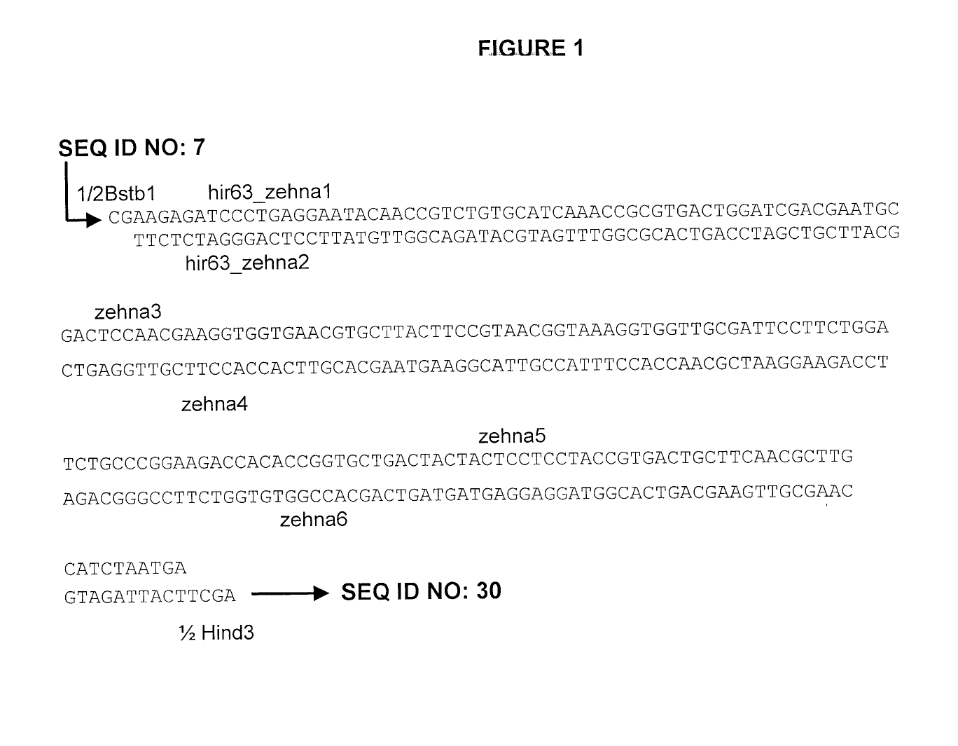

The three codons translated into amino acids in the sequence SEQ ID NO: 1 mark the transition from hirudin to TAP (FIG. 1).

In the DNA coding for the bifunctional fusion protein (SEQ ID NO: 7), the oligonucleotides were arranged such that three blocks hir63-zehna1 and hir63-zehna2, zehna3 and zehna4, and also zehna5 and zehna6 were formed as sense and antisense strands, which can hybridize with one another. In this case, 5′ projecting ends resulted, which in each case hybridized with the projecting end of the next block and were thus ligated. FIG. 1 illustrates this scheme. The projecting ends at the beginning and at the end of the sequence coding for TAP represent in each case one half of the recognition sites for the restriction enzymes Bstb1 and Hind3. These sites were used for cloning the DNA shown in FIG. 1 (SEQ ID NO: 7).

For the preparation of the DNA shown in FIG. 1, 1 μg each of the 6 oligonucleotides were combined in 1 ml of H₂O and then incubated at 94° C. for 10′ and then at 65° C. for 20′. At the end of the hybridization reaction, the mixture was transferred to an ice bath. An aliquot of 150 μl was taken from the batch and employed in a T4 ligase reaction.

The reaction products were concentrated by means of ethanol precipitation and separated from one another on an 8% strength PAA gel. The DNA band corresponding to the expected size of about 200 bp of the DNA illustrated in FIG. 1 (SEQ ID NO: 7) was excised and isolated from the piece of gel. After elution and purification, the fragment was inserted into the plasmid pK152, opened using Bstb1 and Hind3, in a T4 ligase reaction. Competent cells of the strain E. coli MC1061 were transformed with the ligation products, isolated from transformant plasmid DNA and characterized. The inserted DNA fragment from one clone was identified as that of SEQ ID NO: 7 by means of sequence analysis. The plasmid DNA of this clone served as a starting material for further cloning. This plasmid contained the DNA that codes for the desired hirudin-TAP fusion protein.

The Bstb1/Hind3 fragment was reisolated from the plasmid and inserted into the plasmid pD2B in a T4 DNA ligase reaction. Competent cells of the strain E. coli K12 MC1061 (Sambrook et al. “Molecular Cloning”, Old Spring Habor Laboratory Press (1989)) were transformed with the ligation mixture and isolated from transformant plasmid DNA for characterization. In parallel, a storage plate was prepared from the transformants characterized by means of plasmid analysis. Starting from this plate, expression experiments, such as that described in Example 3 of European Patent No. EP 0 549 915, were carried out.

The cells were centrifuged off after expression and the clear supernatants were assayed for hirudin and TAP activity. The control used was an expression supernatant of recombinant E. coli MC1061 cells, which had been transformed using the plasmid pD2B. It was observed that the hirudin activity developed in the supernatants from the control experiment and from the expression of the bifunctional fusion protein was comparatively high. The inhibition constant of the factor Xa was determined to be in the nanomolar range, while in the control experiment null or only slight inhibition was observed. These results show that the bifunctional fusion protein displays both hirudin and TAP activity and that the E. coli variants were surprisingly able to secrete this protein in active form.

### Example 4

### Construction of Hir₍₁₋₆₅₎-Asp Pro- TAP₍₁₋₆₀₎

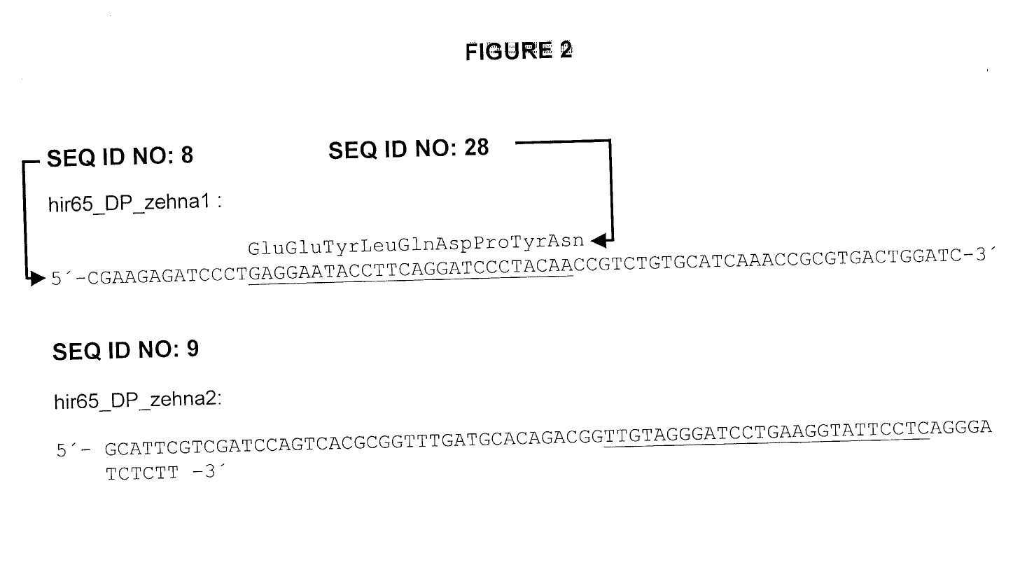

This example describes the preparation and expression of a bifunctional fusion protein, which includes the complete sequence of hirudin and of TAP. Both proteins were separated from one another by a bridge member (spacer) of the form Asp-Pro. For construction, a procedure according to Example 3 was used. Here, however, the oligonucleotides having the sequences SEQ ID NO: 1 (hir63_zehna1) and SEQ ID NO: 2 (hir63_zehna2) were replaced by the oligonucleotides having the sequences SEQ ID NO: 8 (hir65_DP_zehna1) and SEQ ID NO: 9 (hir65_DP_zehna2) (cf. FIG. 2).

After expression, a yield of bifunctional fusion protein comparable to Example 3 results, which acts both like hirudin and like TAP.

### Example 5

### Construction of Hir₍₁₋₆₃₎-Ala Ile Glu Gly Arg- TAP₍₁₋₆₀₎ (Gly34)

The example describes the construction of a hybrid protein, which contains alanine in position 64 of the hirudin sequence instead of leucine, the amino acid 65—glutamine—was deleted, and carries the amino acid glycine in position 34 of the TAP protein instead of aspartic acid. None of the mutations has been described for the respective individual protein. The two fusion components are separated by the recognition site Ile Glu Gly Arg of factor Xa protease.

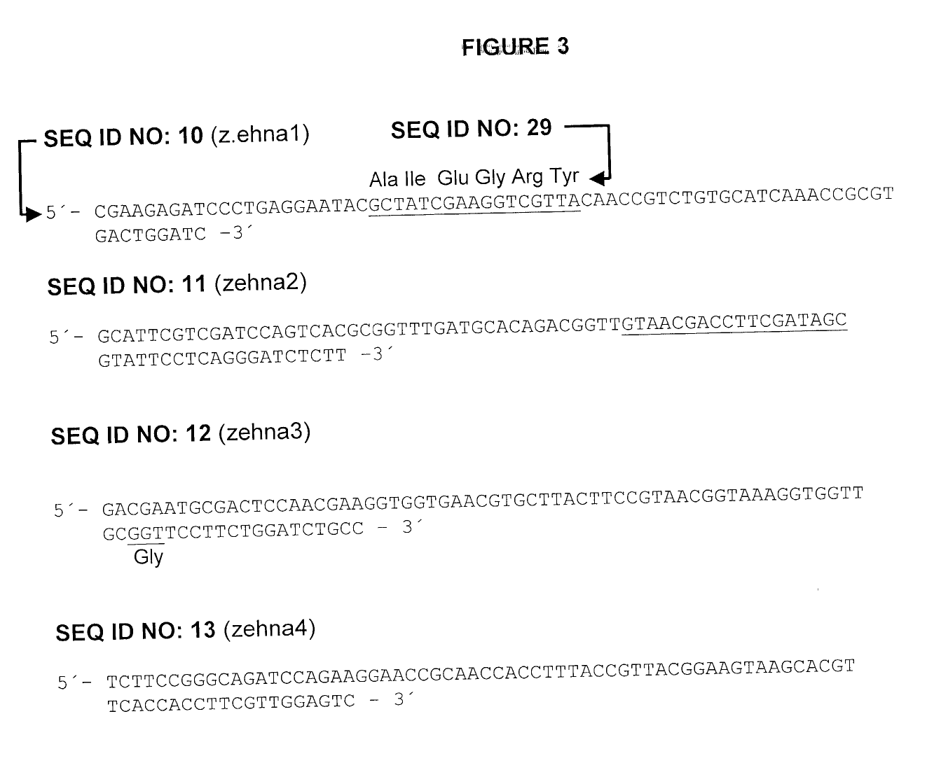

For construction of the DNA sequence encoding the protein, a procedure according to Example 3 was used. Four new oligonucleotides, however, were needed (cf. FIG. 3.)

After expression, a yield of hybrid protein comparable to Example 3 results, which acts both like hirudin and like TAP. Based on the measured hirudin activity, the inhibition constant characterizing the TAP activity lies in the nanomolar range.

### Example 6

This example describes the construction of a plasmid, which codes for a hirudin-TAP derivative that corresponds to the sequence described in Example 5, but carries alanine instead of leucine at the N terminus in the hirudin. To this end, the plasmid pCM7053 described in European Patent No. EP 0 448 093 was opened using the restriction enzymes BamH1 and Hind3 and ligated with the BamH1 hirudin-TAP-Hind3 fragment isolated from the plasmid constructed in Example 5. The resulting plasmid now codes for the protein Ala-Hir₍₂₋₆₃₎-Ala Ile Glu Gly Arg TAP₍₁₋₆₀₎ (Gly34). The protein was actively expressed in yields comparable to Leu-hirudin variants.

The present invention may be embodied in other specific forms. The described embodiments are to be considered in all respects as illustrative only and not restrictive.

All patents, patent applications, and publications cited in the instant application are incorporated by reference.

TABLE 1

| Signal sequence | Primary structure | SEQ ID NO: |
|---|---|---|
| Control: cgtase-Ala-hirudin | MKRNRFFNTSAAIAISIALNTFF | 18 |
| | CSMQTIA | |
| External membrane protein/ | MKKTAIALAVALAGFATVAQA | 19 |
| Serratia marcescens | | |
| opRF-Protein/Pseudomonas | MKNTLGLAIGSLIAATSFGVLA | 20 |
| fluorescens | | |
| lambB-Protein/E.coli | MMITLRKLPLAVAVAAGVMS | 21 |
| | AQAMA | |
| Formate reductase/Shewanella | MKKMNLAVCIATLMGTAGLM | 22 |
| putrifaciens | GTAVA | |
| β-Lactamase/pBR322 | MSIQHFRVALIPFFAAFSLPVFA | 23 |
| alk. Phosphatase/E. coli | MKQSTIALALLPLLFTPVTKA | 24 |
| alk. Phosphatase/ | MKQSAIALALLSCLITPVSQA | 25 |
| E. fergusonii | | |
| Cyclodextrin glucotransferase/ | MKSRYKRLTSLALSLSMALGI | 26 |
| Paenibacillus macerans | SLPAWA | |
| Outer membrane protein/ | MSFHHRVFKLSALSLALFSHLSFA | 27 |
| S. typhimurium | | |
| | See Figure 2 | 28 |
| | See Figure 3 | 29 |
| | See Figure 1 | 30 |

We claim:

1. A bifunctional fusion protein formed from hirudin or a variant of hirudin and TAP or a variant of TAP.

2. The bifunctional fusion protein as claimed in claim 1, comprising the hirudin variant [Leu¹, Thr²]-63-desulfatohirudin or a part of this hirudin variant.

3. The bifunctional fusion protein as claimed in claim 2, comprising the amino acids 1-63 from sequence SEQ ID NO: 15.

4. The bifunctional fusion protein as claimed in claim 2, wherein TAP has the sequence SEQ ID NO: 17, or the variant of TAP corresponds to a part of the sequence SEQ ID NO: 17.

5. The bifunctional fusion protein as claimed in claim 4, wherein hirudin or its variant and TAP or its variant are connected via a spacer, and wherein said spacer consists of one or more amino acids.

6. The bifunctional fusion protein as claimed in claim 4, wherein the fusion protein is coupled to a carrier.

7. The bifunctional fusion protein as claimed in claim 6, wherein the carrier is polyethylene glycol (PEG) or dextran.

8. The bifunctional fusion protein as claimed in claim 4, further comprising a signal peptide.

9. The bifunctional fusion protein as claimed in claim 2, wherein hirudin or its variant and TAP or its variant are connected via a spacer, and wherein said spacer consists of one or more amino acids.

10. The bifunctional fusion protein as claimed in claim 2, wherein the fusion protein is coupled to a carrier.

11. The bifunctional fusion protein as claimed in claim 10, wherein the carrier is polyethylene glycol (PEG) or dextran.

12. The bifunctional fusion protein as claimed in claim 2, further comprising a signal peptide.

13. The bifunctional fusion protein as claimed in claim 1, comprising the amino acids 1-63 from sequence SEQ ID NO. 15.

14. The bifunctional fusion protein as claimed in claim 13, wherein TAP has the sequence SEQ ID NO: 17, or the variant of TAP corresponds to a part of the sequence SEQ ID NO: 17.

15. The bifunctional fusion protein as claimed in claim 14, wherein hirudin or its variant and TAP or its variants are connected via a spacer, and wherein said spacer consists of one or more amino acids.

16. The bifunctional fusion protein as claimed in claim 14, wherein the fusion protein is coupled to a carrier.

17. The bifunctional fusion protein as claimed in claim 16, wherein the carrier is polyethylene glycol (PEG) or dextran.

18. The bifunctional fusion protein as claimed in claim 14, further comprising a signal peptide.

19. The bifunctional fusion protein as claimed in claim 1, wherein TAP has the sequence SEQ ID NO: 17.

20. The bifunctional fusion protein as claimed in claim 19, wherein the variant of TAP corresponds to a part of the sequence SEQ ID NO: 17.

21. The bifunctional fusion protein as claimed in claim 19, wherein hirudin or its variant and TAP or its variant are connected via a spacer, and wherein said spacer consists of one or more amino acids.

22. The bifunctional fusion protein as claimed in claim 19, wherein the fusion protein is coupled to a carrier.

23. The bifunctional fusion protein as claimed in claim 22, wherein the carrier is polyethylene glycol (PEG) or dextran.

24. The bifunctional fusion protein as claimed in claim 19, further comprising a signal peptide.

25. The bifunctional fusion protein as claimed in claim 1, wherein the variant of TAP corresponds to a part of the sequence SEQ ID NO: 17.

26. The bifunctional fusion protein as claimed in claim 25, wherein hirudin or its variant and TAP or its variant are connected via a spacer, and wherein said spacer consists of one or more amino acids.

27. The bifunctional fusion protein as claimed in claim 25, wherein the fusion protein is coupled to a carrier.

28. The bifunctional fusion protein as claimed in claim 27, wherein the carrier is polyethylene glycol (PEG) or dextran.

29. The bifunctional fusion protein as claimed in claim 25, further comprising a signal peptide.

30. The bifunctional fusion protein as claimed in claim 1, wherein hirudin or its variant and TAP or its variant are connected via a spacer, and wherein said spacer consists of one or more amino acids.

31. The bifunctional fusion protein as claimed in claim 30, wherein the fusion protein is coupled to a carrier.

32. The bifunctional fusion protein as claimed in claim 31, wherein the carrier is polyethylene glycol (PEG) or dextran.

33. The bifunctional fusion protein as claimed in claim 30, further comprising a signal peptide.

34. The bifunctional fusion protein as claimed in claim 1, wherein the fusion protein is coupled to a carrier.

35. The bifunctional fusion protein as claimed in claim 34, wherein the carrier is polyethylene glycol (PEG) or dextran.

36. The bifunctional fusion protein as claimed in claim 34, further comprising a signal peptide.

37. The bifunctional fusion protein as claimed in claim 1, further comprising a signal peptide.

38. A DNA sequence coding for a bifunctional fusion protein selected from the group consisting of:
a bifunctional fusion protein formed from hirudin or a variant of hirudin and TAP or a variant of TAP,
a bifunctional fusion protein formed from hirudin or a variant of hirudin and TAP or a variant of TAP, wherein said bifunctional fusion protein comprises the hirudin variant [Leu¹, Thr²]-63-desulfatohirudin or a part of this hirudin variant,
a bifunctional fusion protein formed from hirudin or a variant of hirudin and TAP or a variant of TAP, wherein said bifunctional fusion protein comprises the amino acids 1-63 from sequence SEQ ID NO: 15,
a bifunctional fusion protein formed from hirudin or a variant of hirudin and TAP or a variant of TAP, wherein TAP has the sequence SEQ ID NO: 17,
a bifunctional fusion protein formed from hirudin or a variant of hirudin and TAP or a variant of TAP, wherein the variant of TAP corresponds to a part of the sequence SEQ ID NO: 17,
a bifunctional fusion protein formed from hirudin or a variant of hirudin and TAP or a variant of TAP, wherein hirudin or its variant and TAP or its variant are connected via a spacer, and wherein said spacer consists of one or more amino acids, and
a bifunctional fusion protein formed from hirudin or a variant of hirudin and TAP or a variant of TAP, wherein said bifunctional fusion protein further comprises a signal peptide.

39. A plasmid comprising a DNA sequence as claimed in claim 38.

40. The plasmid comprising a DNA as claimed in claim 39, further comprising a promoter sequence suitable for the expression of said DNA sequence.

41. A cell comprising
a DNA as claimed in claim 38,
a plasmid comprising a DNA sequence as claimed in claim 38, or
a plasmid comprising a DNA sequence as claimed in claim 38, wherein said plasmid further comprises a promoter sequence suitable for the expression of said DNA sequence.

42. A process for the preparation of a bifunctional fusion protein comprising:
incorporating a DNA sequence, which codes for the bifunctional fusion protein, into a cell, and
expressing the DNA sequence in the cell,
wherein said bifunctional fusion protein is selected from the group consisting of:
a bifunctional fusion protein formed from hirudin or a variant of hirudin and TAP or a variant of TAP,
a bifunctional fusion protein formed from hirudin or a variant of hirudin and TAP or a variant of TAP, wherein said bifunctional fusion protein comprises the hirudin variant [Leu¹, Thr²]-63-desulfatohirudin or a part of this hirudin variant,
a bifunctional fusion protein formed from hirudin or a variant of hirudin and TAP or a variant of TAP, wherein said bifunctional fusion protein comprises the amino acids 1-63 from sequence SEQ ID NO: 15,
a bifunctional fusion protein formed from hirudin or a variant of hirudin and TAP or a variant of TAP, wherein TAP has the sequence SEQ ID NO: 17,
a bifunctional fusion protein formed from hirudin or a variant of hirudin and TAP or a variant of TAP, wherein the variant of TAP corresponds to a part of the sequence SEQ ID NO: 17,
a bifunctional fusion protein formed from hirudin or a variant of hirudin and TAP or a variant of TAP wherein hirudin or its variant and TAP or its variant are connected via a spacer, and wherein said spacer consists of one or more amino acids, and
a bifunctional fusion protein formed from hirudin or a variant of hirudin and TAP or a variant of TAP, wherein said bifunctional fusion protein further comprises a signal peptide.

43. The process as claimed in claim 42, wherein the cell is a bacterial cell or a yeast cell.

44. The process as claimed in claim 42, wherein the cell is an E. coli cell.

45. The process as claimed in claim 42, further comprising purification of the bifunctional fusion protein from the medium or cell supernatant.

46. The process as claimed in claim 45, wherein the cell is a bacterial cell or a yeast cell.

47. The process as claimed in claim 46, wherein the cell is an E. coli cell.

48. The process as claimed in claim 45, further comprising freeze-drying the bifunctional fusion protein.

49. The process as claimed in claim 48, wherein the cell is a bacterial cell or a yeast cell.

50. The process as claimed in claim 49, wherein the cell is an E. coli cell.

51. A pharmaceutical composition comprising a bifunctional fusion protein, wherein said bifunctional fusion protein is selected from the group consisting of:
a bifunctional fusion protein formed from hirudin or a variant of hirudin and TAP or a variant of TAP,
a bifunctional fusion protein formed from hirudin or a variant of hirudin and TAP or a variant of TAP, wherein said bifunctional fusion protein comprises the hirudin variant [Leu¹, Thr²]-63-desulfatohirudin or a part of this hirudin variant,
a bifunctional fusion protein formed from hirudin or a variant of hirudin and TAP or a variant of TAP, wherein said bifunctional fusion protein comprises the amino acids 1-63 from sequence SEQ ID NO: 15,
a bifunctional fusion protein formed from hirudin or a variant of hirudin and TAP or a variant of TAP, wherein TAP has the sequence SEQ ID NO: 17,
a bifunctional fusion protein formed from hirudin or a variant of hirudin and TAP or a variant of TAP, wherein the variant of TAP corresponds to a part of the sequence SEQ ID NO: 17,
a bifunctional fusion protein formed from hirudin or a variant of hirudin and TAP or a variant of TAP, wherein hirudin or its variant and TAP or its variant are connected via a spacer, and wherein said spacer consists of one or more amino acids, and
a bifunctional fusion protein formed from hirudin or a variant of hirudin and TAP or a variant of TAP, wherein said bifunctional fusion protein further comprises a signal peptide.

52. The pharmaceutical composition as claimed in claim 51, further comprising a suitable pharmaceutical carrier.

53. The pharmaceutical composition as claimed in claim 52, further comprising other additives.

54. A process for the production of the pharmaceutical composition as claimed in claim 51 comprising,
preparing the bifunctional fusion protein, and
mixing said bifunctional fusion protein with a suitable pharmaceutical carrier.

55. The process for the production of a pharmaceutical composition as claimed in claim 54, further comprising adding other additives to the mixture.

56. A nasal spray comprising a bifunctional fusion protein, wherein said bifunctional fusion protein is selected from the group consisting of:
a bifunctional fusion protein formed from hirudin or a variant of hirudin and TAP or a variant of TAP,
a bifunctional fusion protein formed from hirudin or a variant of hirudin and TAP or a variant of TAP, wherein said bifunctional fusion protein comprises the hirudin variant [Leu¹, Thr²]-63-desulfatohirudin or a part of this hirudin variant,
a bifunctional fusion protein formed from hirudin or a variant of hirudin and TAP or a variant of TAP, wherein said bifunctional fusion protein comprises the amino acids 1-63 from sequence SEQ ID NO: 15,
a bifunctional fusion protein formed from hirudin or a variant of hirudin and TAP or a variant of TAP, wherein TAP has the sequence SEQ ID NO: 17,
a bifunctional fusion protein formed from hirudin or a variant of hirudin and TAP or a variant of TAP, wherein the variant of TAP corresponds to a part of the sequence SEQ ID NO: 17,
a bifunctional fusion protein formed from hirudin or a variant of hirudin and TAP or a variant of TAP, wherein hirudin or its variant and TAP or its variant are connected via a spacer, and wherein said spacer consists of one or more amino acids, and
a bifunctional fusion protein formed from hirudin or a variant of hirudin and TAP or a variant of TAP, wherein said bifunctional fusion protein further comprises a signal peptide.

57. The nasal spray as claimed in claim 56, further comprising a suitable pharmaceutical carrier.

58. The nasal spray as claimed in claim 56, further comprising other additives.

59. A process for the production of a nasally administrable pharmaceutical composition comprising
preparing a bifunctional fusion protein, and
mixing said bifunctional fusion protein with a suitable pharmaceutical carrier,
wherein said bifunctional fusion protein is selected from the group consisting of:
a bifunctional fusion protein formed from hirudin or a variant of hirudin and TAP or a variant of TAP,
a bifunctional fusion protein formed from hirudin or a variant of hirudin and TAP or a variant of TAP, wherein said bifunctional fusion protein comprises the hirudin variant [Leu¹, Thr²]-63-desulfatohirudin or a part of this hirudin variant,
a bifunctional fusion protein formed from hirudin or a variant of hirudin and TAP or a variant of TAP, wherein said bifunctional fusion protein comprises the amino acids 1-63 from sequence SEQ ID NO: 15,
a bifunctional fusion protein formed from hirudin or a variant of hirudin and TAP or a variant of TAP, wherein TAP has the sequence SEQ ID NO: 17,
a bifunctional fusion protein formed from hirudin or a variant of hirudin and TAP or a variant of TAP, wherein the variant of TAP corresponds to a part of the sequence SEQ ID NO: 17,
a bifunctional fusion protein formed from hirudin or a variant of hirudin and TAP or a variant of TAP, wherein hirudin or its variant and TAP or its variant are connected via a spacer, and wherein said spacer consists of one or more amino acids, and
a bifunctional fusion protein formed from hirudin or a variant of hirudin and TAP or a variant of TAP, wherein said bifunctional fusion protein further comprises a signal peptide.

60. The process for the production of a nasally administrable pharmaceutical composition as claimed in claim 59, further comprising adding other additives to the mixture.

61. The process for the production of a nasally administrable pharmaceutical composition as claimed in claim 59, further comprising freeze-drying the mixture.
